(19) United States
(12) Patent Application Publication
(10) Pub. No.: US 2009/0010989 A1
(43) Pub. Date: Jan. 8, 2009

(54) Coating For Implants and Implants With Improved Osteointegration, and Manufacturing Method

(75) Inventor: Tor Peters, Schaffhausen (CH)

Correspondence Address: KNOBBE MARTENS OLSON & BEAR LLP, 2040 MAIN STREET, FOURTEENTH FLOOR, IRVINE, CA, 92614, US

(73) Assignee: N0Labs AB, Heisingborg (SE)

(21) Appl. No.: 12/065,601

(22) PCT Filed: Feb. 13, 2006
(86) PCT No.: PCT/EP2006/050903

§ 371 Date: Sep. 10, 2008

Related U.S. Application Data
(60) Provisional application No. 60/716,192, filed on Sep. 12, 2005.

(51) Int. Cl. A61F 2/04; A61K 9/10; B05D 1/00; A61K 33/00

(52) U.S. Cl. 424/423; 424/484; 424/718; 427/2.14

(57) ABSTRACT

A coating on an implant, said implant being intended for implantation in/on an implantation area, is provided. The coating comprises nitric oxide (NO) for obtaining an anti-viral, anti-fungal, and anti-bacterial effect, and for promotion of osteo-integration of the implant, bone healing, bone growth, and wound healing at said implantation area. A nitric oxide (NO) eluting polymer is integrated with a carrier material, such that said carrier material, in use, regulates and controls the elution of a therapeutic dosage of nitric oxide (NO). An implant and a kit of implants, comprising said coating are also provided. Furthermore, a manufacturing method for the implant is disclosed.

### FIELD OF THE INVENTION

This invention pertains in general to the field of a coating of an implant, said implant being configured for surgical treatment of fractures, deformities, tumour diseases, replacement of tissue, such as bone, and promotion of osteo-integration and wound-healing of the implant, said coating involving the use of nitric oxide (NO). More particularly the present invention pertains to a kit of such coated implants.

### BACKGROUND OF THE INVENTION

In the field of implant surgery, surgeons implant a wide variety of metallic, ceramic, and polymeric materials into patients, such as humans or animals. Surgeons use these kind of implants for orthopaedic purposes, such as treatment of fractures, treatment of deformities, tumour diseases, and replacement of tissue, such as bone, but also in other fields of implantation, such as cosmetic surgery, reconstructive surgery, wire leads, heart surgery, such as heart valve surgery, aneurysm clips, and dental surgery.

A problem associated with insertion of implants is viral and bacteriological infection, caused by virus, fungi, and/or bacteria that get access to the tissue in the vicinity of the inserted implant, when the body of the patient is opened, or when a wound is inflicted during trauma. It is also possible that the implant in itself carries virus, fungi, or bacteria.

Also, the body of the patient, in which the implant has been inserted, recognises implants as foreign objects, possibly leading to local and systemic reactions. Thus, a problem in prior art is osteo-integration of the implants.

Even if bone is hard and strong enough to support the weight of our bodies, it is by no means an unchangeable tissue. Living cells account for about 15% of the weight of compact bone, and these cells are engaged in an unceasing process of remodelling. One class of cells (osteoclasts) destroys old bone matrix while another (osteoblasts) deposits new bone matrix. This mechanism provides for continuous turnover and replacement of the bone matrix in the interior of the bone through which it can adapt to the load it bears. This is also a prerequisite to successful osteo-integration of implants.

It is known that nitric oxide (NO) provides an alternative to conventional therapies, such as antibiotics. Nitric oxide is a highly reactive molecule that is involved in many cell functions. In fact, nitric oxide plays a crucial role in the immune system and is utilized as an effector molecule by macrophages to protect itself against a number of pathogens, such as fungi, viruses, bacteria etc., and general microbial invasion. This improvement of healing is partly caused by NO inhibiting the activation or aggregation of blood platelets, and also by NO causing a reduction of inflammatory processes at the site of an implant.

NO is also known to have an anti-pathogenic, especially an anti-viral, effect, and furthermore NO has an anti-cancerous effect, as it is cytotoxic and cytostatic in therapeutic concentrations, i.e. it has among other effects tumoricidal and bacteriocidal effects. NO has for instance cytotoxic effects on human haematological malignant cells from patients with leukaemia or lymphoma, whereby NO may be used as a chemotherapeutic agent for treating such haematological disorders, even when the cells have become resistant to conventional anti-cancer drugs. This anti-pathogenic and anti-tumour effect of NO is taken advantage of by the present invention, without having adverse effects as for instance many anti-cancer drugs.

However, due to the short half-life of NO, it has hitherto been very hard to treat viral, bacteria, virus, fungi or yeast infections with NO. This is because NO is actually toxic in high concentrations and has negative effects when applied in too large amounts to the body. NO is actually also a vasodilator, and too large amounts of NO introduced into the body will cause a complete collapse of the circulatory system. On the other hand, NO has a very short half-life of fractions of a second up to a few seconds, once it is released. Hence, administration limitations due to short half-life and toxicity of NO have been limiting factors in the use of NO in the field of anti-pathogenic and anti-cancerous treatment so far.

In recent years research has been directed to polymers with the capability of releasing nitrogen oxide when getting in contact with water. Such polymers are for example polyalkyleneimines, such as L-PEI (Linear PolyEthyleneImine) and B-PEI (Branched PolyEthyleneImine), which polymers have the advantage of being biocompatible.

Other example for NO eluting polymers are given in U.S. Pat. No. 5,770,645, wherein polymers derivatized with at least one —NOₓ group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of permanently implanted medical devices such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner to tissues and organs to aid the healing process and to prevent injury to tissues at risk of injury. Electrospun nano-fibers of linear poly(ethylenimine) diazeniumdiolate deliver therapeutic levels of NO to the tissues surrounding a medical device while minimizing the alteration of the properties of the device. A nanofiber coating, because of the small size and large surface area per unit mass of the nanofibers, provides a much larger surface area per unit mass while minimizing changes in other properties of the device.

However, the meaning of “controlled” in the context of U.S. Pat. No. 6,737,447 is only directed to the fact that nitric oxide is eluted from the coating during a period of time. Therefore, the interpretation of “controlled” in respect of U.S. Pat. No. 6,737,447 is different from the meaning of “regulating” in the present invention. “Regulate”, according to the present invention is intended to be interpreted as the possibility to vary the elution of nitric oxide to thereby achieve different elution profiles.

US 2004/0131753 discloses a coating for medical devices, which coating provides NO delivery by using nanofibers of L-PEI. The technical effect of US 2004/0131753 is that the released NO will help prevent platelet aggregation and smooth muscle cell proliferation. It is unclear how the elution of NO is initiated in this application. The elution of nitric oxide from the coating according to US 2004/0131753 is not regulated in any way. Furthermore, US 2004/0131753 is totally silent about improved osteointegration.

U.S. Pat. No. 6,270,779 describes biocompatible metallic medical devices with silanized surfaces coupled to nucleophilic residues that release therapeutic amounts of nitric oxide to specific sites within a mammalian body. Thus, the medical devices according to this patent are all metallic, and the method of manufacturing them are in need of a silanization step. The elution of nitric oxide from the metallic surface according to U.S. Pat. No. 6,270,779 is not regulated in any way. Furthermore, U.S. Pat. No. 6,270,779 is totally silent about improved osteointegration.

WO 03/026717 describes a method for preparing a nitric oxide-releasing substrate, such as medical devices, similar to those mentioned in U.S. Pat. No. 6,270,779. Thus, the elution of nitric oxide from the substrate according to WO 03/026717 is not regulated in any way. Furthermore, WO 03/026717 is totally silent about improved osteointegration.

US 2003/083739 discloses a system for treating vascular in-stent restenosis, with silanized medical devices. The elution of nitric oxide from the silanized device according to US 2003/083739 is not regulated in any way. Furthermore, US 2003/083739 is totally silent about improved osteointegration.

U.S. Pat. No. 5,770,645 discloses medical devices coated with nitric oxide eluting polymers for reducing platelet deposition and restenosis. The elution of nitric oxide from the device according to U.S. Pat. No. 5,770,645 is not regulated in any way. Furthermore, U.S. Pat. No. 5,770,645 is totally silent about improved osteointegration.

Pulfer, S. K., et al., “Incorporation of nitric oxide-releasing crosslinked polyethyleneimine microspheres into vascular grafts”, Journal of Biomedical Materials Research, Wiley, New York, N.Y., US, vol. 37, no. 2, November 1997, discloses site-specific delivery of nitric oxide by entrapping nitric oxide releasing polyethyleneimine microspheres in the pores of a vascular graft. The effects obtained with these grafts are inhibition of platelet aggregation, smooth-muscle cell proliferation, and elimination of need for systemic anticoagulants. The elution of nitric oxide from the polymer according to this article is not regulated in any way. Furthermore, this article is totally silent about improved osteointegration.

Shabani, M., et al., “Enhancement of wound repair with a topically applied nitric-oxide releasing polymer”, Wound Repair and Regeneration, Mosby-Year Book, St. Louis, Mo., US, vol. 4, no. 3, 1 Jul. 1996, discloses a PEI-C NONOate polymer for topical use. The elution of nitric oxide from the polymer according to this article is not regulated in any way. Furthermore, this article is totally silent about improved osteointegration.

Bohl Masters, K. S., et al., “Effects of nitric oxide releasing poly(vinyl alcohol) hydrogel dressings on dermal wound healing in diabetic mice” Wound Repair and Regeneration, Mosby-Year Book, St. Louis, Mo., US, vol. 10, no. 5, 2002, describes in vitro and in vivo responses to a novel hydrogel, manufactured by ultraviolet light-initiated polymerization from poly(vinyl alcohol) with a NO donor covalently coupled to the polymer backbone, that produces therapeutic levels of NO. This is a dermally applied polymer, hence nothing is indicated about osteointegration. Furthermore, the elution of nitric oxide from the hydrogel according to this article is not regulated in any way.

Thus, the disclosure is both silent concerning an improvement of present technology in respect of a coating of an NO eluting polymer on implants to provide an anti-bacterial, anti-fungi, and anti-viral effect, by elution of nitric oxide NO, to thereby also obtain an improved osteointegration. Furthermore, the disclosure is silent concerning regulating and/or controlling the elution of nitric oxide from such coatings.

Thus, it would be appreciated to provide a way of obtaining an anti-viral, anti-fungal, and anti-bacterial effect, while simultaneously obtaining promotion of osteo-integration of the implant, bone healing, bone growth, and wound healing.

### SUMMARY OF THE INVENTION

Accordingly, the present invention preferably seeks to mitigate, alleviate or eliminate one or more of the above-identified deficiencies in the art and disadvantages singly or in any combination and solves among others at least the problems mentioned above, at least partly by providing a coating, an implant, and a kit of implants, according to the appended patent claims.

According to one aspect of the invention, a coating is provided, which coating allows for anti-viral, anti-fungal, and anti-bacterial effect, and promotion of osteo-integration of the implant, bone healing, bone growth, and wound healing, on an implant. Said coating comprises a nitric oxide (NO) eluting polymer, such that a therapeutic dose of nitric oxide is eluted from said nitric oxide eluting polymer, allowing for anti-viral, anti-fungal, and anti-bacterial effect, and promotion of osteo-integration of the implant, bone healing, bone growth, and wound healing.

According to another aspect of the invention, an implant is provided, which implant has at least partly said coating.

According to still another aspect of the invention a kit of said implants is provided.

The present invention has at least the advantage over the prior art that it provides target exposure of a tissue or organ in the vicinity of an implant to NO, whereby an increased circulation in the tissue or organ area, anti-viral, anti-fungal, and anti-bacterial effect, and promotion of osteo-integration of the implant, bone healing, bone growth, and wound healing, while not developing resistance against the active pharmaceutical substance, pain etc, simultaneously are obtained.

### BRIEF DESCRIPTION OF THE DRAWING

These and other aspects, features and advantages of which the invention is capable of will be apparent and elucidated from the following description of embodiments of the present invention, reference being made to the accompanying drawing, in which

FIG. 1 is an illustration of one example of an implant according to an embodiment of the present invention.

### DESCRIPTION OF EMBODIMENTS

The following description focuses on embodiments of the present invention applicable to a coating on implants, which coating allows for anti-viral, anti-fungal, and anti-bacterial effect, and promotion of osteo-integration of the implant, bone healing, bone growth, and wound healing.

The patient according to the embodiments may be a human or animal, such as mammals selected from the group consisting of cat, dog, horse, cattle etc.

With regard to nitric oxide (nitrogen monoxide, NO), its physiological and pharmacological roles have attracted much attention and thus have been studied. NO is synthesized from arginine as the substrate by nitric oxide synthase (NOS). NOS is classified into a constitutive enzyme, cNOS, which is present even in the normal state of a living body and an inducible enzyme, iNOS, which is produced in a large amount in response to a certain stimulus. It is known that, as compared with the concentration of NO produced by cNOS, the concentration of NO produced by iNOS is 2 to 3 orders higher, i.e. 100 to 1000 folded higher, and that iNOS produces an extremely large amount of NO.

In the case of the generation of a large amount of NO as in the case of the production by iNOS, it is known that NO reacts with active oxygen to attack exogenous microorganisms and cancer cells, but also to cause inflammation and tissue injury. On the other hand, in the case of the generation of a small amount of NO as in the case of the production by cNOS, it is considered that NO takes charge of various protective actions for a living body through cyclic GMP (cGMP), such as vasodilator action, improvement of the blood circulation, antiplatelet-aggregating action, antibacterial action, anticancer action, acceleration of the absorption at the digestive tract, renal function regulation, neurotransmitting action, erection (reproduction), learning, appetite, and the like. Heretofore, inhibitors of the enzymatic activity of NOS have been examined for the purpose of preventing inflammation and tissue injury, which are considered to be attributable to NO generated in a large amount in a living body. However, the promotion of the enzymatic activity (or expressed amount) of NOS (in particular, cNOS) has not been examined for the purpose of exhibiting various protective actions for a living body by promoting the enzymatic activity of NOS and producing NO appropriately.

It has now been shown that NO is an important local mediator of bone cell activity. Changes in the mechanical forces acting on bone lead to adaptive remodelling of the bone. NO is an important signalling molecule on mature bone tissue, triggering the adaptive response.

Osteoblasts and osteclasts both produce and respond to NO; low doses of NO support and higher doses inhibit osteoclast and osteoblast function.

All three types of NOS are involved in the development and homeostasis of bone tissue. Basal low-level NO synthesis by eNOS and nNOS stimulates osteoblasts and osteoclasts, respectively, and is essential for their function. Lack of eNOS results in reduced bone formation and bone volume. ENOS-deficient osteoblasts also show weaker response to the growth factor TGF-beta that is necessary for the requirement of osteoblasts to remodelling sites. nNOS-deficiency, on the other hand, show defective bone turn-over.

Exogenous NO in still higher concentrations inhibits bone resorption by suppressing the formation and activity of osteoclasts.

The present invention takes advantage of these facts and therefore presents an unexpected effect in respect of osteo-integration of implants by using the NO eluting coating on implants.

In recent years research has been directed to polymers with the capability of releasing nitrogen oxide when getting in contact with water. Such polymers are for example polyalkyleneimines, such as L-PEI (Linear PolyEthyleneImine) and B-PEI (Branched PolyEthyleneImine), which polymers have the advantage of being biocompatible.

The polymers employed in embodiments of the present invention may be manufactured by electro spinning, air spinning, gas spinning, wet spinning, dry spinning, melt spinning, and gel spinning. Electro spinning is a process by which a suspended polymer is charged. At a characteristic voltage a fine jet of polymer releases from the surface in response to the tensile forces generated by interaction by an applied electric field with the electrical charge carried by the jet. This process produces a bundle of polymer fibres, such as nano-fibres. This jet of polymer fibres may be directed to a surface to be treated.

Furthermore, U.S. Pat. No. 6,382,526, U.S. Pat. No. 6,520,425, and U.S. Pat. No. 6,695,992 disclose processes and apparatuses for the production of such polymeric fibres. These techniques are generally based on gas stream spinning, also known within the fiber forming industry as air spinning, of liquids and/or solutions capable of forming fibers. Gas stream spinning is suited for producing devices according to certain embodiments of the invention.

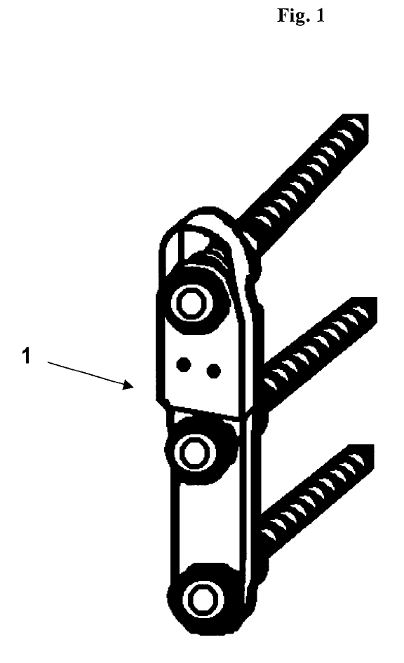

In an embodiment of the invention an NO eluting polymer is electro spun onto an implant. The implant may, according to different embodiments, for example be a temporary, a permanent, or biodegradable implant. Temporary implants are implants that are removed after a certain time period of implantation. For instance a per se known device 1 comprising screws and/or plates, as shown in FIG. 1, is fixed to a fractured bone across the fracture site thereof. The device is however provided with a coating eluting NO during a certain time after implantation of the device 1. Thus for instance osteo-integration is promoted and the fracture bone heals faster than in the case where device 1 does not have such an advantageous coating. After healing is at least partly achieved, e.g. when the bone fracture has healed to sufficient stability, the temporary device 1 is removed by surgery. Alternative embodiments of biodegradable implants have the ability to break down, safely and relatively quickly, by biological means, into the raw materials of nature and disappear from the body where they were implanted in. In the latter case, the coating eluting NO during a certain time after implantation is also biodegradable or at least biocompatible.

The implant according to an embodiment of the invention is an orthopaedic implant, such as (i) a hip joint, (ii) screws, cannulated screws, nails, intramedullary nails, and plates intended to join or attach bone fragments, pieces, or parts with each other, (iii) external fixators, (iv) implants intended for treatment of degenerative instabilities, fractures, tumours, and deformities in respect of the spine, (v) craniomaxillofacial implants intended for treatment of fractures, reconstruction, and correction of deformities, of mandible, mid-face, or skull.

In other embodiments the implant may be chosen from the group: 1) dental implants and sealing caps, that are temporarily put over e.g. a titanium screw before an artificial tooth is mounted on the titanium screw, 2) internal and external wound closure, 3) cosmetic surgery, 4) reconstructive surgery, 5) wire leads, 6) heart surgery, such as heart valve surgery, 7) aneurysm clips, 8) ear implants, such as drainage tubes through the eardrum during infection, 9) infusion systems, such as cytostatic infusion systems, 10) stomia systems, such as colostomy, tracheotomy tubes and systems, and 11) tear channel implants.

After the implant, according to above, has been coated with an NO eluting polymer the implant may be mounted, placed, or applied on the area in need of implantation. When the coated implant is in place and gets in contact with the inevitable moisture or water in the body, in which the implant has been implanted, the NO eluting polymer in the coating of the implant starts to elute NO.

In another embodiment of the present invention the implant is partially covered with NO eluting polymer. This embodiment may for example be used when only a part of the implant that is inside the subject body, such as in respect of fixation means for holding a head, vertebra, or knee in a position, which position, for some reason, needs regulation during the healing process. It is of course also within the scope of the present invention to cover the entire implant in these cases with the NO eluting coating, but it would be more economically to only cover the part in contact with the subject body, i.e. a target area.

The elution of NO then brings about an anti-viral, anti-fungal, and anti-bacterial effect, and promotion of osteo-integration of the implant, bone healing, bone growth, and wound healing on the target area.

Three important factors in controlling and regulating the elution of nitric oxide from a nitric oxide eluting polymer are how quickly a proton donor comes in contact with the nitric oxide releasing polymer, such as a diazoliumdiolate group, the acidity of the environment surrounding the nitric oxide eluting polymer, and the temperature of the environment surrounding the nitric oxide releasing polymer (higher temperature promotes elution of nitric oxide).

In one embodiment the NO eluting polymer is co-spun together with a carrier material, such as another polymer, or other polymers, onto the implant. “Co-spun” in the present context is intended to be interpreted as spun, as a polymer mixture, together with the NO eluting polymer, either by air-spinning, electro spinning, wet spinning, dry spinning, air spinning, melt spinning, or gel spinning. This/these other polymer/polymers may for example be chosen from the group: polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

In one embodiment of the present invention a nitric oxide eluting polymer, such as L-PEI-NO, is mixed with a carrier polymer to slow down or prolong the elution of nitric oxide. Also, in another embodiment, the nitric oxide eluting polymer may be mixed with more than one carrier polymer, whereby be elution or release may be tailor made to fit specific needs. Such a need may for example be a low elution during a first period of time, when the environment of the nitric oxide eluting polymer is hydrophobic, and a faster elution during a second period of time, when the environment of the nitric oxide eluting polymer has been altered to be more hydrophilic. This may for example be accomplished by using biodegradable polymers, whereby a low elution during a first period of time is obtained, after which, when the hydrophobic polymer has been dissolved, the hydrophilic polymer provides a higher elution of nitric oxide. Thus, a more hydrophobic carrier polymer will give a slower elution of nitric oxide, since the activating proton donor, such as water or body fluid, will penetrate the carrier polymer slower. On the other hand, a hydrophilic polymer acts the opposite way. One example of an hydrophilic polymer is polyethylene oxide, and one example of an hydrophobic polymer is polystyrene. These carrier polymers may be mixed with the nitric oxide eluting polymer and then electrospun to suitable fibers. The skilled person in the art knows which other polymers may be used for similar purposes. FIG. 4 illustrates two elution profiles (NO concentration vs. time) for two different polymer mixtures; a nitric oxide eluting polymer mixed with a hydrophilic carrier polymer in an acidic environment (A), and a nitric oxide eluting polymer mixed with a hydrophobic carrier polymer in a neutral environment (B).

In one embodiment this carrier polymer is substituted by another material with hydrophobic or hydrophilic properties. Therefore, the term “carrier material” in the present context should be interpreted to include carrier polymers and other materials with hydrophilic or hydrophobic properties.

In another embodiment of the present invention the elution of nitric oxide from a nitric oxide eluting polymer, such as L-PEI-NO, is influenced by the presence of protons. This means that a more acidic environment provides a quicker elution of nitric oxide. By activating the nitric oxide eluting polymer, or mixture of nitric oxide eluting polymer and carrier material, with an acidic fluid, such as an ascorbic acid solution, the elution of nitric oxide may be accelerated.

The carrier polymers and carrier materials mentioned above may affect other characteristics than the regulation of nitric oxide elution. Examples of such characteristic is mechanical strength.

In respect of the carrier polymers or carrier materials, the NO-eluting polymer may be integrated in, spun together with, or spun on top of, any of these materials in all of the embodiments of the present invention. This spinning includes electro spinning, air spinning, dry spinning, wet spinning, melt spinning, and gel spinning. In this way, one may manufacture fibers of a polymer mixture, comprising a nitric oxide eluting polymer and a carrier polymer, or a carrier material, with predefined nitric oxide eluting characteristics. These characteristics may be tailor made for different elution profiles in different applications.

Other example for NO eluting polymers are given in U.S. Pat. No. 5,770,645, wherein polymers derivatized with at least one —NOX group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of permanently implanted medical devices such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner.

However, the meaning of “controlled” in the context of U.S. Pat. No. 6,737,447 is only directed to the fact that nitric oxide is eluted from the coating during a period of time, i.e that the nitric oxide not is eluted all in once. Therefore, the interpretation of “controlled” in respect of U.S. Pat. No. 6,737,447 is different from the meaning of “regulating” in the present invention. “Regulate or control”, according to the present invention is intended to be interpreted as the possibility to vary the elution of nitric oxide to thereby achieve different elution profiles.

A polymer comprising an O-nitrosylated group is also a possible nitric oxide eluting polymer. Thus, in one embodiment of the present invention, the nitric oxide eluting polymer comprises diazeniumdiolate groups, S-nitrosylated and O-nitrosylated groups, or any combinations thereof.

In still another embodiment of the present invention said nitric oxide eluting polymer is a poly(alkyleneimine)diazeniumdiolate, such as L-PEI-NO (linear poly(ethyleneimine)diazeniumdiolate), where said nitric oxide eluting polymer is loaded with nitric oxide through the diazeniumdiolate groups and arranged to release nitric oxide at a treatment site.

Some other examples of a suitable nitric oxide eluting polymer are selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

In still another embodiment of the present invention the nitric oxide eluting polymer may be a O-derivatized NONOate. This kind of polymer often needs an enzymatic reaction to release nitric oxide.

Other ways of describing polymers, which may be suitable as nitric oxide eluting polymer, is polymers comprising secondary amine groups (═N—H), such as L-PEI, or have a secondary amine (═N—H) as a pendant, such as aminocellulose.

The nitric oxide eluting polymer may comprise a secondary amine, either in the backbone or as a pendant, as described previously. This will make a good nitric oxide eluting polymer. The secondary amine should have a strong negative charge to be easy to load with nitric oxide. If there is a ligand close to the secondary amine, such as on a neighbour atom, such as a carbon atom, to the nitrogen atom, with higher electronegativity than nitrogen (N), it is very difficult to load the polymer with nitric oxide. On the other hand, if there is a electropositive ligand close to the secondary amine, such as on a neighbour atom, such as a carbon atom, to the nitrogen atom, the electronegativity of the amine will increase and thereby increase the possibility to load the nitric oxide elution polymer with nitric oxide.

In an embodiment of the present invention the nitric oxide polymer may be stabilized with a salt. Since the nitric oxide eluting group, such as a diazeniumdiolate group, usually is negative, a positive counter ion, such as a cation, may be used to stabilize the nitric oxide eluting group. This cation may for example be selected from the group comprising any cation from group 1 or group 2 in the periodic table, such as Na⁺, K⁺, Li⁺, Be²⁺, Ca²⁺, Mg²⁺, Ba²⁺, and/or Sr²⁺. Different salts of the same nitric oxide eluting polymer have different properties. In this way a suitable salt (or cation) may be selected for different purposes. Examples of cationic stabilized polymers are L-PEI-NO-Na, i.e. L-PEI diazeniumdiolate stabilized with sodium, and L-PEI-NO-Ca, i.e. L-PEI diazeniumdiolate stabilized with calcium.

Another embodiment of the present invention comprises mixing the nitric oxide eluting polymer, or a mixture of the nitric oxide eluting polymer and a carrier material, with an absorbent agent. This embodiment provides the advantage of an accelerated elution of nitric oxide since the polymer, or polymer mixture, via the absorbent agent, may take up the activating fluid, such as water or body fluid, much faster. In one example 80% (w/w) absorbent agent is mixed with the nitric oxide eluting polymer, or mixture of nitric oxide eluting polymer and carrier material, and in another embodiment 10 to 50% (w/w) absorbent agent is mixed with the nitric oxide eluting polymer, or mixture of nitric oxide eluting polymer and carrier material.

Since the elution of nitric oxide is activated by a proton donor, such as water, it may be an advantage to keep the nitric oxide eluting polymer, or mixture of nitric oxide eluting polymer and carrier material, in contact with said proton donor. If an indication requires an elution of nitric oxide during a prolonged period of time, a system is advantageous, which presents the possibility to keep the proton donor in contact with the nitric oxide eluting polymer, or mixture of nitric oxide eluting polymer and carrier material. Therefore, in still another embodiment of the present invention, the elution of nitric oxide may be regulated by adding an absorbent agent. The absorbent agent absorbs the proton donor, such as water, and keeps the proton donor in close contact with the nitric oxide eluting polymer during prolonged periods of time. Said absorbent agent may be selected from the group comprising polyacrylates, polyethylene oxide, carboxymethylcellulose, and microcrystalline cellulose, cotton, and starch. This absorbent agent may also be used as a filling agent. In this case said filling agent may give the nitric oxide eluting polymer, or mixture of said nitric oxide eluting polymer and a carrier material, a desired texture.

In still another embodiment the NO eluting polymer, according to above, is ground or milled into nano-particles or micro-spheres. These nano-particles or micro-spheres are then applied on the implant by any convenient method, which method is known by the skilled artisan, such as gluing with a glue that not is dissolvable in the body environment of the implant. It is also possible to mix or encapsulate fibres, nano-particles, or micro-spheres of NO eluting polymer with other polymers, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. When the fibres, nano-particles, or micro-spheres of NO eluting polymer, according to this embodiment, gets in contact with the moisture or water in the implantation area, elution of NO starts and an anti-viral, anti-fungal, and anti-bacterial effect is obtained. This embodiment presents the advantage of controlling or regulating the time span of NO release from the implant, by the mixing of other polymers that do not elute NO.

In the context of the present invention the term “encapsulating” is intended to be interpreted as fixating the nitric oxide eluting polymer in a three dimensional matrix such as a foam, a film, a nonwoven mat of nano-fibers, fibers, other materials with the capability to fixate the NO eluting polymer, or enclosing the nitric oxide eluting polymer in any suitable material.

In one embodiment the nitric oxide eluting polymer, such as powder, nano-particles or micro-spheres, can be incorporated in foam. The foam may have an open cell structure, which facilitates the transport of the proton donor to the nitric oxide eluting polymer. The foam can be of any suitable polymer such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these, or latex. This foam is then applied on the device, to obtain improved osteointegration.

In still another embodiment the NO eluting polymer is integrated in a film of another suitable polymer (polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these) film, which film then is glued on the implant under the restrictions mentioned above. When these film, including NO eluting polymer, gets in contact with the moisture or water in the implantation area, elution of NO starts and an anti-viral, anti-fungal, and anti-bacterial effect, and promotion of osteo-integration of the implant, bone healing, bone growth, and wound healing is obtained.

In another embodiment the nano-particles, or micro-spheres according to above, may be integrated in a soluble film that disintegrates on the implantation area, in order to elute NO at the area of interest when the soluble film gets in contact with the moisture or water in the implantation area.

The device elutes nitric oxide (NO) from said eluting polymer in a therapeutic dose, such as between 0.001 to 5000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm. The concentration may vary widely depending on where the concentration is measured. If the concentration is measured close to the actual NO eluting polymer the concentration may be as high as thousands of ppm, while the concentration inside the tissue in this case often is considerably lower, such as between 1 to 1000 ppm.

The NO-eluting polymers in the coating may be combined with silver, such as hydroactivated silver. The integration of silver in the devices gives the anti-microbial and anti-viral effect an extra boost. Preferably the silver is releasable from the devices in the form of silver ions. The integration of silver in the device may present several advantages. One example of such an advantage is that the silver may keep the device in itself free from bacteria or viruses, while the nitric oxide eluting polymer elutes the therapeutic dosage of nitric oxide to the target site.

In yet another embodiment of the present invention the NO-eluting coating is acting as a booster for drug eluting implants, e.g. pharmaceuticals, vitamins, nicotin, nitroglycerin, etambutol, Non-Steroidal Anti-Inflammatory Drugs (NSAID), such as diclofenac, ibuprofen, aspirin, naproxen, COX-2 inhibitors, choline magnesium trisalicylate, diflunisal, salsalate, fenoprofen, flurbiprofen, ketoprofen, oxaprozin, indomethacin, sulindac, tolmetin, meloxicam, piroxicam, meclofenamate, mefenamic acid, nabumetone, etodalac, ketorolac, celecoxib, valdecoxib, and rofecoxib; steroids, such as cortisone, prednisone, methylprednisolone, prednisolone, vitamin D, estrogen, cholestrol, beclomethasone, flunisolide, fluticasone, triamcinolone, desonide, clobetasol, alclometasole, desoximetasone, betamethasone, halcinonide and dexamethasone; pain reliefs, such as motrin, feldene, naprosyn, lidocaine, and prilocaine; and other substances, such as indinavirsulfate, finasteride, aprepitant, montelukast sodium, alendronate sodium, rofecoxib, rizatriptan benzoate, simvastatin, finasteride, ezetimibe, caspofungin acetate, ertapenem sodium, dorzolamide hydrochloride, timolol maleate, losartan potassium, and hydrochlorotiazide; etc. This embodiment presents a coating with the advantage of combining two treatments, of significant value, in one treatment.

The device may be manufactured by, for example electro spinning of for example L-PEI. L-PEI is then charged at a characteristic voltage, and a fine jet of L-PEI releases as a bundle of L-PEI polymer fibres. This jet of polymer fibres may be directed to a surface to be treated. The surface to be treated may for example be any suitable material. The electro spun fibres of L-PEI then attach on said material and form a coating/layer of L-PEI on the device according to the invention.

It is of course possible to electro spin the other NO-eluting polymers, according to above, on the implant while still being inside the scope of the present invention as defined by the appended claims.

In one embodiment the NO-eluting polymers employed in the coating are electro spun in such way that pure NO-eluting polymer fibres may be obtained.

Gas stream spinning, air-spinning, wet spinning, dry spinning, melt spinning, and gel spinning, of said NO-eluting polymers onto the implant is also within the scope of the present invention.

The manufacturing process presents the advantages of large contact surface of the NO-eluting polymer fibres or micro particles with the area to be covered with the coating, effective use of NO-eluting polymer, and a cost effective way of coating the implant.

The invention may be implemented in any suitable form. The elements and components of the embodiments according to the invention may be physically, functionally, and logically implemented in any suitable way. Indeed, the functionality may be implemented in a single unit, in a plurality of units, or as part of other functional units.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A coating of an implant, said implant being configured to be implanted in/on an implantation area,
comprising a nitric oxide eluting polymer, configured to elute Nitric Oxide (NO), for obtaining an anti-viral, anti-fungal, and anti-bacterial effect, and configured to promote osteo-integration of the implant, bone healing, bone growth, and wound healing at said implantation area, wherein said coating covers said implant at least partly
characterized in that
said nitric oxide (NO) eluting polymer is integrated with a carrier material, such that said carrier material, in use, regulates and controls the elution of said therapeutic dosage of nitric oxide (NO).

2. The coating according to claim 1, wherein said elution of nitric oxide is chosen to support osteoclast and osteoblast function.

3. The coating according to claim 1, wherein said nitric oxide (NO) eluting polymer comprises diazeniumdiolate groups, S-nitrosylated groups, and O-nitrosylated groups, or any combination these.

4. The coating according to claim 1, wherein said nitric oxide (NO) eluting polymer is L-PEI (linear polyethyleneimine), loaded with nitric oxide (NO) through said diazeniumdiolate groups, S-nitrosylated groups, or O-nitrosylated groups, or any combination these, arranged for release of the nitric oxide (NO) at said implantation area in, or on, a body of a human or animal.

5. The coating according to claim 1, wherein said nitric oxide eluting polymer is selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

6. The coating according to claim 1, wherein said coating is at least partly disintegrable when subjected to a proton donor.

7. The coating according to claim 1, wherein said polymer is in form of nano-particles or micro-spheres.

8. Coating according to claim 7, wherein said nano-particles, or micro-spheres, are encapsulated in suitable material, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these.

9. Coating according to claim 1, wherein said carrier material is selected from the group comprising polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these.

10. Coating according to claim 1, wherein said coating comprises silver, configured for exposure of said area.

11. Coating according to claim 1, wherein said coating is configured to act as a booster for other active ingredients chosen from the group consisting of pharmaceuticals, vitamins, nicotin, nitroglycerin, Non-Steroidal Anti-Inflammatory Drugs, steroids, and/or pain reliefs.

12. The coating according to claim 1, wherein said nitric oxide eluting polymer comprises a secondary amine in the backbone or a secondary amine as a pendant.

13. The coating according to claim 12, wherein a positive ligand is located on the neighbour atom to the secondary amine.

14. The coating according to claim 1 or 9, comprising an absorbent agent.

15. The coating according to claim 14, wherein said absorbent agent is selected from the group comprising polyacrylate, polyethylene oxide, Carboxy Methyl Cellulose (CMC), microcrystalline cellulose, cotton, or starch, or any combinations thereof.

16. The coating according to claim 1, 9, or 14, comprising a cation, said cation stabilizing the nitric oxide eluting polymer.

17. The coating according to claim 16, wherein said cation is selected from the group comprising Na⁺, K⁺, Li⁺, Be²⁺, Ca²⁺, Mg²⁺, Ba²⁺, and/or Sr²⁺, or any combinations thereof.

18. An implant, comprising the coating according to claim 1.

19. The implant according to claim 18, wherein said implant is an orthopaedic implant, such as (i) a hip joint, (ii) screws, cannulated screws, nails, intramedullary nails, and plates intended to join or attach bone fragments, pieces, or parts with each other, (iii) external fixators, (iv) implants intended for treatment of degenerative instabilities, fractures, tumours, and deformities in respect of the spine, or (v) craniomaxillofacial implants intended for treatment of fractures, reconstruction, and correction of deformities, of mandible, mid-face, or skull.

20. The implant according to claim 18 or 19, wherein said implant is selected from the group: 1) dental implants and sealing caps, that are temporarily put over the titanium screw before an artificial tooth is mounted on the titanium screw, 2) internal and external wound closure, 3) cosmetic surgery, 4) reconstructive surgery, 5) wire leads, 6) heart surgery, such as heart valve surgery, 7) aneurysm clips, 8) ear implants, such as drainage tubes through the eardrum during infection, 9) infusion systems, such as cytostatic infusion systems, 10) stomia systems, such as colostomy, tracheotomy tubes and systems, and 11) tear channel implants.

21. A kit of implants according to claim 18, wherein said implants comprise the coating according to claim 1.

22. The kit according to claim 21, wherein said kit comprises screws and plates intended to join or attach bone fragments, pieces, or parts with each other.

23. The kit according to claim 21, wherein said kit comprises temporary implants, bio-degradable and/or non-bio-degradable implants.

24. A process for applying a coating according to claim 1 on an implant according to claim 11, comprising:
selecting a plurality of nitric oxide eluting polymeric particles, preferably nano fibres, nano particles or micro spheres, and a carrier material and
deploying said nitric oxide eluting particles and carrier material as a coating on said implant,
wherein said deploying comprises electro, air, gas stream, wet, dry, melt, gel spinning of said particles.

25. A manufacturing process for an implant according to claim 18, comprising:
selecting a nitric oxide (NO) eluting polymer configured to elute a therapeutic dosage of nitric oxide (NO) when used for said,
selecting a carrier material, which carrier material is configured to regulate and control the elution of said therapeutic dosage of nitric oxide (NO) for in use of the implant obtaining an anti-viral, anti-fungal, and anti-bacterial effect and promoting osteo-integration of the implant, bone healing, bone growth, and wound healing at an implantation area thereof,
incorporating the NO-eluting polymer with said carrier material into an nitric oxide (NO) eluting material, such that said carrier material, in use of said device, regulates and controls the elution of said therapeutic dosage of nitric oxide (NO), and
deploying said nitric oxide eluting material as a coating covering said implant at least partly, such that the implant is configured to expose the implantation area to said nitric oxide when said NO-eluting polymer in use elutes nitric oxide (NO).

26. The manufacturing process according to claim 25,
wherein said deploying comprises electro spinning, air spinning, gas spinning, wet spinning, dry spinning, melt spinning, or gel spinning of NO-eluting polymer.

27. The manufacturing process according to claim 25 or 26, wherein said selecting said nitric oxide (NO) eluting polymer comprises selecting a plurality of nitric oxide (NO) eluting polymeric particles, preferably nano fibres, nano particles or micro spheres.

28. The manufacturing process according to claim 25 or 26, wherein said incorporating said NO-eluting polymer with said carrier material comprises integrating said NO-eluting polymer in said carrier material, spinning said NO-eluting polymer together with said carrier material, or spinning said NO-eluting polymer on top of said carrier material, in order to predefine nitric oxide eluting characteristics of said implant.

29. The manufacturing process according to claim 25, further comprising integrating silver in said device.

30. Use of a nitric oxide (NO) eluting polymer for the manufacture of a coating on an implant, said implant being intended for implantation in/on an implantation area, wherein
nitric oxide is loaded to said coating, which coating elutes nitric oxide (NO) from said eluting polymer in a non-toxic dose when used in/on said implantation area for obtaining an anti-viral, anti-fungal, and anti-bacterial effect, and for promotion of osteo-integration of the implant, bone healing, bone growth, and wound healing at said implantation area.

31. Use according to claim 30, wherein said non-toxic dose is 0.001 to 5000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm.
